(19) United States
(12) Patent Application Publication
(10) Pub. No.: US 2023/0411009 A1
(43) Pub. Date: Dec. 21, 2023

(54) SYSTEM AND METHOD FOR ZERO BURDEN UNIVERSAL SCREENING ALGORITHMS FOR COMPLEX DISEASES

(71) Applicant: University of Chicago, Chicago, IL (US)

(72) Inventors: Ishanu Chattopadhyay, Oak Lawn, IL (US); Dmytro Onishchenko, Chicago, IL (US)

(21) Appl. No.: 18/337,780

(22) Filed: Jun. 20, 2023

Related U.S. Application Data
(60) Provisional application No. 63/353,236, filed on Jun. 17, 2022.

(51) Int. Cl. G16H 50/20; G16H 10/60; G16H 50/30

(52) U.S. Cl. CPC G16H 50/20; G16H 70/60; G16H 50/30; G16H 10/60

(57) ABSTRACT

A method including receiving a plurality of electronic health records stored in a database and partitioning the plurality of electronic health records in a first set of plurality of electronic health records and a second set of plurality of health records is disclosed. The method includes, for each electronic health record of the first and second sets of the plurality of electronic health records, generating a plurality of data streams, and in accordance with the generated data streams corresponding to the respective group of related disorders, inferring probabilistic finite state automaton (PFSA) models corresponding to a positive cohort and a control cohort for a specific health condition. The method includes determining a respective sequence likelihood defect of an electronic health record data of a new patient to match one of the inferred PFSA models for determining a likelihood of the new patient to acquire the specific health condition.

### CROSS REFERENCE TO RELATED APPLICATION

This application claims priority to and the benefit of U.S. Provisional Patent Application Ser. No. 63/353,236, entitled “ZERO BURDEN UNIVERSAL SCREENING ALGORITHMS FOR COMPLEX DISEASES,” filed Jun. 17, 2022, the contents of which are incorporated herein in their entirety.

### STATEMENT REGARDING FEDERALLY SPONSORED RESEARCH & DEVELOPMENT

This invention was made with support in part by the Defense Advanced Research Project Agency (DARPA) project number HR00111890043/P00004. The government has certain rights in the invention.

### TECHNICAL FIELD

This disclosure relates generally to evaluating risk factors of a patient to have ailments of a complex disease based on the patient's historical health records, and, in particular, to screening algorithms for early screening for complex diseases, without the patient undergoing any new test.

### BACKGROUND

Artificial intelligence and machine learning are transforming workplaces around the world, and they have the potential to radically alter the field of healthcare. Traditionally, physicians have relied on physical examination of patients coupled with results from laboratory tests or imaging methods to diagnose a patient experiencing symptoms or to flag a patient at risk for disease. Such methodology is invasive, time consuming and can be expensive. Moreover, accurate diagnosis may be affected by the physicians training and experience and may be limited by the availability of known illness-related features. The introduction of computer-assisted diagnosis is expected to address some of these issues. However, to date, computer-assisted medicine is often targeted at replacing the physician, making diagnoses based on the same, limited number of illness-related features available to the physician.

The present disclosure uses an individual's general medical history to predict the individual's risk for specific medical conditions. The disclosed methods do not require any additional testing, nor do they require any specific data relating to the individual. Instead, the disclosed methods subject the individual's entire available medical history to machine learning algorithms trained to identify feature patterns impossible to discern by merely reading a patient's history. By comparing a patients' feature pattern learned from millions of features across thousands of medical histories, such algorithms are able to flag those individuals at risk for various medical conditions. Examples of such conditions include, cut are not limited to, major adverse cardiac events (MACE), Alzheimer's disease, and Idiopathic pulmonary fibrosis (IPF).

In many patients undergoing total hip or total knee arthroplasty, depending on multiple cardiac comorbidities, the chances that a patient may suffer from major adverse cardiac events (MACE) including, for example, myocardial infarction and cardiac arrest, may significantly vary. Currently, a revised cardiac risk index (RCRI) is a widely used pre-operating risk calculator that uses existing cardiovascular (CVD) comorbidities and surgical procedural risk to determine perioperative risk for MACE. However, RCRI generally does not consider many currently known CVD risk factors or cannot predict risk to patients who are not yet formally diagnosed to have CVD comorbidities.

Similarly, Alzheimer's disease, which is the most common cause of Dementia in about 60-80% of cases, generally progresses over years or decades before a clinical diagnosis can be made. However, accurate screening for Alzheimer's disease and related dementia (ADRD) is limited by the current diagnostics/prognostic modalities. Currently known imaging or cerebrospinal fluid testing for evaluating a patient's risk for ADRD is expensive, invasive, and sometimes inaccessible. Neuropsychological testing instruments like the Montreal Cognitive Assessment (MOCA) generally has good diagnostic accuracy and some prognostic utility in identifying mild cognitive impairment (MCI) and mild Alzheimer's disease, using MOCA in primary care setting pose significant challenges, in particular, when used in additional locales or languages, their efficacy in predicting future diagnosis may be suspected.

Idiopathic pulmonary fibrosis (IPF) is an irreversible, progressive, debilitating, and lethal fibrosing interstitial lung disease, and timely, efficient, and confident diagnosis of IPF is recognized as a major public health challenge. Reliable early diagnosis of IPF is generally hindered due to most common clinical symptoms of the disease, which may be attributed to age and/or more common cardio-respiratory diseases. In addition, early-stage detection of IPF is exceedingly difficult using the currently known diagnosis methods. Similarly, early and confirmed diagnosis of autism spectrum disorder (ASD) is difficult due to lengthy evaluations, cost of care, etc.

While only a few complex diseases are listed herein, diagnosis for many complex health situations is difficult, invasive, and expensive. Accordingly, non-invasive and more accurate methods for early detection of many complex health situations are needed. The present disclosure addresses these needs.

### BRIEF DESCRIPTION

In one aspect, a computer-implemented method is disclosed. The method includes receiving a plurality of electronic health records stored in a database. Each electronic health record of the plurality of electronic health records corresponds with a patient of a plurality of patients. The method includes partitioning the plurality of electronic health records in a first set of plurality of electronic health records and a second set of plurality of health records based on a gender of each patient of the plurality of patients. The first set of plurality of electronic health records includes electronic health records of males, and the second set of plurality of electronic health records includes electronic health records of females. The method includes generating a plurality of data streams for each electronic health record of the first and second sets of the plurality of electronic health records. Each data stream of the plurality of data streams corresponds to a respective group of related disorders. The respective group of related disorders is associated with a subset of diagnostic codes of a set of diagnostic codes. The method includes, in accordance with the generated data streams corresponding to the respective group of related disorders, inferring a first probabilistic finite state automaton (PFSA) model corresponding to a positive cohort for a specific health condition and a second PFSA model corresponding to a control cohort for the specific health condition. The method includes receiving an electronic health record data of a new patient, and determining a respective sequence likelihood defect of the electronic health record data to match the first PFSA model and the second PFSA model for the specific health condition. The method includes determining that the new patient has a higher likelihood to acquire the specific health condition in accordance with the respective sequence likelihood defect of the electronic health record data to match the first PFSA model being higher than the respective sequence likelihood defect of the electronic health record data to match the second PFSA model, and determining that the new patient has lower likelihood to acquire the specific health condition, in accordance with the respective sequence likelihood defect of the electronic health record data to match the second PFSA model being higher than the respective sequence likelihood defect of the electronic health record data to match the first PFSA model.

In another aspect, a system including at least one memory and at least one processor is disclosed. The system performs operations of the method according to an aspect, as described herein.

### BRIEF DESCRIPTION OF THE DRAWINGS

FIG. 1 is an example block diagram of a distributed computing system including a client device and an application server, in accordance with some embodiments.

FIG. 2 is an example flow-chart of method operations for predicting a specific health condition risk for a patient without requiring the patient undergoing any new laboratory work or new tests, in accordance with some embodiments.

### DETAILED DESCRIPTION OF THE DISCLOSURE

The following detailed description illustrates embodiments of the disclosure by way of example and not by way of limitation. It is contemplated that the disclosure has general application in the medical field, and in particular, diagnosis of diseases and complex health situations.

Embodiments of the present disclosure are directed to zero-burden (ZB) approaches to rapid, universal, and/or early screening for complex diseases in primary care settings. The ZB approach for diagnosis relies on information collected from electronic health records (EHRs) of a patient and evaluating the EHRs of the patient using one or more machine-learning (ML) algorithms. The one or more ML algorithms are trained using EHRs of a number of patients for a predictive EHR analytics approach based on longitudinal pattern discovery in medical history. Using the ZB approach, as described herein, no new bloodwork or laboratory test is required, and predictions may be made at about 95% specificity for Autism Spectrum Disorder, Cerebral Palsy, Alzheimer's Disease, Major Adverse Cardiac Event (MACE) after total knee or hip replacement, Idiopathic Pulmonary Fibrosis (IPF), chronic kidney disease (CKD), and First Manic Switch (Bipolar Disorder), and many others. In some cases, using the ZB approach as described herein, certain cancers, for example, bladder cancer, thyroid cancer, pancreatic cancer, and/or advanced melanoma may be predicted about one year in advance of clinical diagnosis using at least two years of EHR of a patient.

In some embodiments, the ZB approach as described herein, may not require a manually curated fixed set of input features involving laboratory tests, patient demographic information to populate inputs for a ML algorithm training and validation. Training and/or validating a ML algorithm may put an unnecessary burden on the patient, caregivers, and the healthcare system. Instead, the ZB approach relies on algorithmic pattern discovery in EHR databases. In particular, one or more ML algorithms for the ZB approach may be trained to identify complex characteristics of comorbidity incidence, timing, sequence, and synchronism, which presage various diagnoses and outcomes.

A patient's EHR may include a variable length history of individual diagnostic histories corresponding to order, frequency, and comorbid interactions between diseases. Accordingly, each patient's individual EHR and related diagnostic histories may be important for assessing future risk of a target phenotype. A phenotype, as described herein, may be observable characteristics in an individual which result from the expression of genes of the individual. Future risk of a target phenotype may he assessed by analyzing patient-specific diagnostic code sequences as represented in each patient's medical history. From each patient's medical history as laid out in each patient's EHR, a set of stochastic categorical time-series may be identified.

By way of a non-limiting example, in some embodiments, a set of stochastic categorical time-series may include a time-series corresponding to a particular group of related disorders. From the set of stochastic categorical time-series, which forms individual data streams, stochastic models may be inferred based upon the individual data streams. The inferred stochastic models, in some examples, may be a special class of Hidden Markov Models (HMMs), and may be referenced in the present disclosure as Probabilistic Finite State Automata (PFSA). Inference algorithms used to derive PFSA models may be different from algorithms used in generating HMMs.

In some embodiments, PFSA models may be derived for two different classes. For example, a first class of PFS :A models may be related to a positive cohort class and a second class of PFSA models may be related to a control cohort class. Using the PFSA models corresponding to the positive cohort class and the control cohort class, a probability of whether a new patient's diagnostic history is more likely to arise from the positive cohort class or the control cohort class may be identified to help predict how likely the new patient may suffer from the particular complex disease or related disorders.

In some embodiments, PFSA models may be prepared using health history or EHRs of patients that include at least a specific number of years of health history or EHRs. All diagnostic codes that were recorded in the patient's health history or EHRs for at least the specific number of years may be used for training a predictive pipeline. The specific number of years may be referenced herein as an inference window. Using the PFSA models, a diagnostic code at 2 years from the end of the inference window may be predicted. In some examples, a diagnostic code at some time in the future (e.g., 1, 2, or 4 years) from the end of the inference window may also be predicted. For individuals in the control cohort class, it is expected that no diagnostic code appears in the next predictive window (e.g., next 2 or 4 years from the end of the inference window). By way of a non-limiting, a number of years at the end of the inference window may be arbitrarily determined. The number of years at the end of the inference window may, thus, depend on a prediction window, or a number of years in advance for prediction before clinical diagnosis of a disease or other related health condition.

In some embodiments, all diagnostic codes included in a patient's EHRs, or health history may be partitioned into a specific number of non-overlapping categories, for example, 26 different non-overlapping categories. Each non-overlapping category may include, or may be defined by, a set of diagnostic codes. By way of a non-limiting example, each diagnostic code of the set of diagnostic code may be a diagnostic code as defined by the Ninth Revision of International Classification of Diseases (ICD9).

A total number of diagnostic codes for a male may be different from a total number of diagnostic codes for a female. In some examples, a total number of unique diagnostic codes for a male and a female may be around 17,554 and 19,209, respectively, based on ICD9 and ICD10. ICD10, as described herein, may refer to General Equivalence Mappings (GEMS) equivalents. By way of a non-limiting example, in some embodiments, transforming all diagnostic codes from EHRs into broad non-overlapping categories may reduce a total number of diagnostic codes for handling and/or analysis, and, therefore, may also improve statistical power. Additionally, or alternatively, non-overlapping categories may be selected from top-level ICD9 categories. In one example, all ICD9 diagnostic codes associated with infections may be grouped together in a single category by disregarding a pathogen which causes an infection and a particular body part in which the infection occurred.

In some embodiments, each patient's health history may include a plurality of diagnostic codes, and each diagnostic code of the plurality of diagnostic codes may have a corresponding timestamp identifying a date and/or time when the particular diagnostic code may be known and/or added to a patient's health history or HER. Alternatively, from a patient's EHRs, the plurality of diagnostic codes and their corresponding timestamp may be identified. The plurality of diagnostic codes and their corresponding timestamp for each patient may be presented as a timeseries, for examples, as a sequence of (t₁, x₁), (t₂, x₂), . . . , (tₘ, xₘ), and so on, where t₁, t₂, tₘ represent timestamps, and x₁, x₂, and xₘ represent diagnostic codes respective to t₁, t₂, tₘ, respectively.

In some embodiments, each patient's medical history as a timeseries may be mapped to a three-alphabet categorical timeseries Z^{k} corresponding to each disease category k. In particular, while mapping each patient's medical history as a timeseries corresponding to each disease category, for a unit time period (e.g., a week) when there is no diagnostic code associated with a corresponding disease category, a value 0 may be used to represent a diagnostic code for that unit time period. In cases, where there exists a diagnostic code associated with the corresponding disease category, a value 1 may be used to represent a diagnostic code for that unit time period. For each unit time period, where there is a diagnostic code present but if the diagnostic code is unrelated to the corresponding disease category, a value 2 may be used to represent a diagnostic code for that unit time period.

From each patient's mapped timeseries corresponding to each disease category as Z^{k}, PFSA models (which are specialized HMMs) may be generated. PFSA models generated based on the mapped timeseries and for each disease category, and a diagnostic status corresponding to a particular disease for which prediction is being sought are individual sample paths. Since as described herein, 26 different broad categories are being considered for PFSA models, a total of 104 different PSFA models may be generated by also factoring in a gender (e.g., male or female) of each patient and whether the patient is a member of positive cohort class or a control cohort class.

In some embodiments, each of the inferred or derived PFSA model may be a direct graph with probability-weighted edges, and may act as an optimal generator of the stochastic process driving the sequential appearance of three alphabet categorical timeseries Z^{k} corresponding to each disease category k. To reliably infer probability of a particular diagnostic code in future related to a specific diseases, for example, a perioperative cardiac event status-type for a new patient, or in other words, to reliably infer likelihood of a diagnostic sequence being generated by the corresponding perioperative cardiac event status-type model, a notion of Kullbeck-Leibler (KL) divergence may be generalized between probability distributions to a divergence between ergodic stationary categorical stochastic processes G, H as shown in an equation below:

$${𝒟}_{KL} ( G \vert \vert H ) = {lim}_{n \to ∞} \frac{1}{n} {\sum }_{x ; \{ x \} = n} {p}_{G} ( x ) log \frac{{p}_{G} ( x )}{{p}_{H} ( x )}$$

In the above equation, P_{G}(x) and P_{H}(x) are probabilities of a sequence x being generated by the processes G and H, respectively. A log-likelihood of a sequence x being generated by a process G may be defined using the following equation:

$$L ( x , G ) = - \frac{1}{\vert x \vert} log {p}_{G} ( x )$$

A particular cohort-type (a positive cohort class or a control cohort class) for an observed sequence x, which is generated by a hidden process G, may be inferred from observations based on the provable relationships, as shown in equations below:

$${lim}_{\vert x \vert \to ∞} L ( x , G ) = ℋ ( G ) \\ {lim}_{\vert x \vert \to ∞} L ( x , H ) = ℋ ( G ) + {𝒟}_{KL} ( G \vert \vert H )$$

In the above equations, the computed likelihood has an additional non-negative contribution from the divergence term when an incorrect generating process is chosen. Accordingly, for a patient, who is eventually going to be diagnosed with, for example, perioperative cardiac event, the patient's disease-specific mapped timeseries Z^{k} corresponding to the patient's health history or EHRs be modeled by the PFSA model in a positive cohort class.

In some embodiments, sequence likelihood defect (SLD) may be computed for the inferred PFSA models corresponding to positive and control cohorts of a disease category j using the following equation:

Δ^{j} [symbol] L(G₀^{j}, x)−L(G^{j}₊, x)→ [symbol] _{KL}(G₀^{j}∥G₊^{j})

Where (SLD, Δ^{j}) represents a sequence likelihood defect, and G₊^{j}, G₀^{j} represents positive and control cohorts, respectively.

Accordingly, using the inferred PFSA models and a patient's individual diagnostic history, a sequence likelihood defect (SLD) may be measured. By way of an example, a higher value of SLD may indicate a higher similarity of diagnostic history of the patient for the particular disease category, for example, perioperative cardiac event, in this case.

In some embodiments, a risk estimation pipeline may operate on patient specific information limited to available diagnostic history for the inference window, as described herein, and without requiring a patient to undergo any new tests or laboratory work. In other words, an estimation of a patient to have risk for a particular disease category may be known from the risk estimation pipeline. An associated confidence value for the patient to have a diagnostic code related to the particular disease category may also be known using the risk estimation pipeline.

In some embodiments, parameters and associated model structures of the risk estimation pipeline may be identified by transforming patient specific data, as learned from the EHRs of the patient, to a set of engineered features and feature vectors. The set of engineered features and feature vectors may be realized on positive and control cohorts. The feature vectors realized on the positive and control cohorts may be used to train a gradient-boosting classifier (or a machine-learning algorithm).

Two training sets may be used to train the gradient-boosting classifier (or the machine-learning algorithm). A first training set may be used to infer one or more machine-learning models, and a second training set may be used to train the gradient-boosting classifier with features derived from the inferred one or more machine-learning models. By way of a non-limiting example, health histories or EHRs of a number of patients may be randomly divided into three different groups for inferring the one or more machine-learning models and training the gradient-boosting classifier.

A first group of patients may be randomly selected, and their health histories or EHRs may be used for feature-engineering (or to infer PFSA models, as described in detail below), a second group of patients may be randomly selected, and their health histories or EHRs may be used for training the machine-learning algorithm. A third group of patients may be randomly selected, and their health histories or EHRs may be used for testing or validation. Each of the first group, second group, and/or the third group may have a different number of patients. By way of an example, out of total 100% patients, 25% of patients may be randomly selected for the first group and/or the second group, and 50% of the patients may be selected for the third group.

In some examples, the inferred PFSA models may be unsupervised machine-learning models for each disease category. The inferred PFSA models may be trained using a training set associated with health histories or EHRs of the second group of patients. The trained machine-learning models may therefore be supervised learning models, which may be tested using health histories or EHRs of the third group of patients.

In some embodiments, a receiver operating characteristics (ROC) curve identifying a plot between a false positive rate (FPR) and a true positive rate (TPR) may also be generated. An area under the ROC curve (AUC) may be used to measure performance of the classifier (or the machine-learning algorithm). A TPR, a FPR, a true negative rate (TNR), a positive predictive value (PPV), and a prevalence value ρ may be defined using the following:

$$TPR = \frac{{t}_{p}}{P} = \frac{{t}_{p}}{{t}_{p} + {f}_{n}} \\ TNR = \frac{{t}_{n}}{N} = \frac{{t}_{n}}{{t}_{n} + {f}_{p}} \\ FOR = 1 - TNR \\ PPV = \frac{{t}_{p}}{{t}_{p} + {f}_{p}} \\ p = \frac{P}{N + P}$$

In the above, tₚ, tₙ, fₚ, and fₙ correspond with true positives, true negatives, false positives, and false negatives, respectively. The TPR may also be referred herein as recall or sensitivity(s), and PPV may also be referenced herein as precision. The TNR may also be referenced herein as specificity (c). A precision-recall curve, or a PPV-sensitivity curve may be defined as a plot between PPV and TPR.

Based on the sensitivity (s) and the specificity (c), PPV may be as:

$$PPV = \frac{{t}_{p} / P}{{t}_{p} / P + ( {f}_{p} / N ) ( N / P )} = \frac{TPR}{TPR + ( ( N - {t}_{n} ) / N ) ( N / P )}$$

In other words, PPV may be represented as shown below:

$$PPV = \frac{s}{s + ( 1 - c ) ( \frac{1}{p} - 1 )}$$

From the above, for a fixed value of sensitivity (s) and specificity (c), the PPV depends on prevalence, such that PPV decreases with a decrease in prevalence, and vice versa. The specificity (c) may be generally selected to a very high value, such as 95%. A selection of a decision threshold value to trade-off a true positive rate and a false positive rate may depend upon a value of specificity (c). By way of an example, the decision threshold value may be desired to be independent of the number of true negatives in general, and in cases where the number of negatives is large in comparison with the number of positives.

In some examples, the decision threshold value may be determined

based on an accuracy and an F1-score, which are represented as:

$$accuracy = \frac{{t}_{p} + {t}_{n}}{{t}_{p} + {f}_{p} + {f}_{n} + {t}_{n}}$$

$$F 1 = \frac{2 {t}_{p}}{2 {t}_{p} + {f}_{p} + {f}_{n}}$$

In the above equations, the F1-score may be the same as the accuracy where the number of true negatives is same as the number of true positives, and therefore, partially correcting a class imbalance.

The ROC curve derived as described herein may be robust to class imbalance, or in other words, the prediction outcomes are independent and the true positives (tₚ) scales linearly with the total number of positives P. As a result, TPR may be presented as:

$$TPR = \frac{{t}_{p}}{P} = \frac{{t}_{p}^{′}}{{P}^{′}}$$

Accordingly, for a different size of the set of positive samples (or negative samples), the ROC curve may remain unchanged. However, the precision-recall curve may be affected by class imbalance, or prevalence value.

In some embodiments, PFSA models may be inferred from a set of input streams. In other words, the PFSA models may be constructed without specifying any structure and/or number of states in an algorithm. All parameters of the PFSA models may be inferred directly from data of the input streams. The input streams here correspond with health histories or EHRs of the first group of patients randomly selected for inferring PFSA models. The data relied upon for inferring PFSA models may be from a single input stream and/or a set of input streams of different length. Each input stream may correspond to a health history of different patient, and similarly each input stream may include health records over a different time period, for example, a different number of years or months,

In the following, one example of how to infer PFSA models is described in accordance with various technical terms and their corresponding representations.

### Probabilistic Finite-State Automaton

In some embodiments, in the PFSA model, in each state, an input alphabet may have a probability to go to any other state. Accordingly, in PFSA, if Σ is a finite alphabet of symbols with size |Σ|, a set of sequences of a length d over Σ may be denoted by Σ^{d}, a set of finite but unbounded sequences over Σ may be denoted by Σ*. Similarly, the Kleene start operation may be denoted by:

Σ*=U_{d=0}^{∞}Σ^{d}

A sequence x of symbols or events (σs) may be represented as x=σ₁σ₂ . . . σₙ, and a length of the sequence x may be represented |x|. An empty sequence may be denoted by λ.

A set of strictly infinite sequences over Σ may be represented by Σ^{ω}. A set of strictly infinite sequences having x as prefix by xΣ^{ω}.

Using the following equation, it may be verified that [symbol] is a semiring over Σ^{ω}. By way of an example, [symbol] may be used to denote a sigma algebra generated by [symbol] .

[symbol]

={xΣ^{ω}: x∈Σ*}∪{∅}

### Stochastic Process Over Σ

A stochastic process over a finite alphabet Σ may be defined as a collection of Σ-valued random variables (xₜ)_{t∈N} indexed by positive integers. In some processes, Xᵢs may, or may not, be independently distributed.

Sequence-induced measure and derivative:

For a process [symbol] , Pᵣ₍ₓ₎ may denote a probability that the process producing a sample path prefixed by x. A measure μₓ induced by a sequence x that belongs to Σ* may be an extension to [symbol] of the premeasure. The premeasure may be defined on the semiring [symbol] by:

$$∀ x , y ∈ {Σ}^{*} , {μ}_{x} ( y {Σ}^{ω} ) {=}^{Δ} \frac{Pr ( xy )}{Pr ( x )} , if Pr ( x ) > 0$$

Here, for any d of N, the d-th order derivative of a sequence x may be written as ϕₓ^{d} and defined as a marginal distribution of μₓ on Σ^{d}. An entry indexed by y may be denoted by ϕₓ^{d}(y). A first-order derivative may be called a symbolic derivative and may be dented Φₓ for short.

### Probabilistic Nerode Equivalence and Casual States

For any pair of sequences x, y belonging to Σ*, x may be equivalent to y and may be written as x˜y, if and only if Pᵣ₍ₓ₎₌P_{r(y)}=0, or μₓ₌μ_{y}.

An equivalence class of a sequence x may be denoted by [x] and may be called as casual state. A cardinality of a set of casual states may be referenced as a probabilistic Nerode index. The casual states may capture how the history of a process may influence the future. Since the probabilistic Nerode equivalence is right invariant, it may give rise to an automaton structure as described herein below.

### Probabilistic Finite-State Automaton (PFSA)

A PFSA G may be defined by a quadruple (Q, Σ, δ, \tilde{π}), where Q is a finite set and Σ is a finite alphabet. Further, δ: Q×Σ−>Σ may be described or referenced as a transition map.

\tilde{π}: Q→P_{z}, where P_{E} may be the space of probability distribution over Σ and may also be referenced as transition probability. An entry of \tilde{π}(q) indexed by σ may be denoted as \tilde{π}(q, σ).

### Transition and Observation Matrices

A transition matrix Π may be a |Q|×|Q| matrix with an indexed q, q′ and written as π_{q, q′} satisfying

$${π}_{q , {q}^{′}} {=}^{Δ} {\sum }_{\{ α ∈ Σ \vert δ ( q , σ ) = {q}^{′} \}} \tilde{π} ( q , σ )$$

An observation matrix \tilde{Π} may be |Q|×|Σ| matrix with the entry by q, σ equaling \tilde{π}(q, σ). Both Π and \tilde{Π} may be stochastic, or in other words, non-negative with rows summing up to 1.

Extension of δ and \tilde{Π} to Σ*:

For any x=σ1σ2 . . . σk, δ(q, x) may be defined recursively by:

δ(q, x) [symbol] δ(δ(q, σ₁ . . . σₖ₋₁), σₖ), δ(q, λ)=q

Also \tilde{π}(q, x) may be recursively defined by:

$$\tilde{π} ( q , x ) {=}^{Δ} {\prod }_{i = 1}^{k} \tilde{π} \{ δ ( q , {σ}_{1} … {σ}_{i - 1} ) , {σ}_{i} \} and \tilde{π} ( q , λ ) = 1.$$

### Strongly Connected PFSA

A PFSA may be strongly connected if an underlying directed graph is strongly connected. A PFSA G that is defined by the quadruple (Q, Σ, δ, \tilde{π}) may be strongly connected if for any pair of distinct states q and q′∈Q, there is an x belonging to Σ* such that δ(q, x)=q′.

By way of an example, all PFSA described herein may be strongly connected. For strongly connected PFSA G, there is a unique probability distribution over Q that satisfies v^{T}Π=v^{T}. This unique probability distribution may be stationary distribution of G and may be denoted by [symbol] G.

### Γ-Expression

By way of an example, information contained in δ and \tilde{Π} may be encoded by a set of |Q|×|Q| matrices as:

$$Γ = \{ {Γ}_{σ} \vert σ ∈ Σ \} , where \\ {Γ}_{σ} {\vert}_{q , {q}^{′}} {=}^{Δ} \{ \begin{matrix}\tilde{π} ( q , σ ) & if δ ( q , σ ) = {q}^{′} , \\ 0 & if otherwise .\end{matrix}$$

Γ_{σ} may be an event-specific transition matrix, with the event being that σ is current the output. Γ_{σ} may be extended to arbitrary x∈Σ* by defining Γₓ=Πᵢ₌₁^{k}Γₒ. with Γ_{λ}=1.

### Sequence Induced Distribution on States

For A PFSA G that is defined by the quadruple (Q, Σ, δ, \tilde{π}) and a distribution [symbol] ₀ on Q, the distribution on Q induced by a sequence x may be given by:

[symbol]

(x)=[[ [symbol] ₀^{T}Γₓ]] with [symbol] (λ)=

[symbol]

An entry indexed by a q∈Q of a vector [symbol] (x) may be written as [symbol] (x, q). When [symbol] = [symbol] , the stationary distribution of G, [symbol] (x), may be written as [symbol] (x).

### Stochastic Process Generated by a PFSA

For a PFSA G that is defined by the quadruple (Q, Σ, δ, \tilde{π}) and [symbol] as a distribution on Q, a Σ-valued stochastic process {Xₜ}_{t∈Σ} generated by G and [symbol] may satisfy that X₁ follows the distribution [symbol] and Xₜ₊₁ follows the distribution [symbol] (X₁ . . . Xₜ) for t∈ [symbol] .

In some examples, [symbol] = [symbol] may be assumed. Further, when a stochastic process is initialized with [symbol] , the process generated by the PFSA G may be stationary and ergodic. In some examples, for the process generated by the PFSA G, ϕₓ= [symbol] (x)^{T}\tilde{Π} when [symbol] (λ)= [symbol] . Here, a symbolic derivative of an empty sequence ϕ_{λ} may be the stationary distribution on the symbols.

### Synchronizable PFSA and Synchronizing Sequence

A synchronizing sequence is a finite sequence that may send an arbitrary state of the PFA to a fixed state. In particular, for a PFSA G that is defined by the quadruple (Q, Σ, δ, \tilde{π}), a sequence [symbol] ∈Σ* is a synchronizing sequence to a state q∈Q if δ(q′, x)=q for all q′∈Q. A PFSA having at least one synchronizing sequence is a synchronizable PFSA. For a sample path generated by a PFSA, a PFSA is synchronizable if a synchronizing sequence transpires in the sample path.

In some examples, a PFSA G over a state set Q, where ε>0, a

sequence x is a ε-synchronizing to a state q∈Q if: ∥ [symbol] (x)−e_{q}∥_{∞}≤ε.

### Equivalence and Irreducibility

Two PFSAs G and H are equivalent if they generate the same stochastic process. A PFSA G is said to be irreducible if not another PDSA may have a smaller state set that is equivalent to state set of the PFSA G.

In some examples, a PFSA that is not synchronizable may be referenced herein as an irreducible PFSA. The irreducible PFSA, as described herein, may always have an ε-synchronizing sequence for some state q for arbitrarily small ε>0. By way of an example, as length increases, sequences produced by PFSA may become uniformly ε-synchronizing. As described herein, these equivalence and irreducibility are underpinning properties for an inference algorithm of PFSA because Φₓ may be used to approximate \tilde{Π}(q) in the case where x are properly prefixed and have a length of certain threshold value. The inference algorithm of PFSA is described below.

### Joint ε-Synchronizing Sequence

For two PFSAs G and H and having state sets Q_{G} and Q_{H}, respectively, and a fixed ε, a sequence x may be referenced as jointly ε-resynchronizing to (q, r)ϵQ_{G}×Q_{H}, if the sequence x is ε-resynchronizing to q and to r simultaneously. Accordingly:

Σ_{ε, (q, r)}^{d} [symbol] {xϵΣ^{d}: x jointly ε-resynchronizing to (q, r)}

### Joint Pair of States

For two PFSAs G and H and having state sets Q_{G} and Q_{H},

respectively, and for a pair of states (q, r)ϵQ_{G}×Q_{H} may be referenced herein as G-joint pair of states if [symbol] _{G}(q, r)>0.

In addition, the followings may also be defined.

$${P}_{G} ( q , r ) {=}^{Δ} {lim}_{d \to ∞} {p}_{G} ( {\sum }_{ε , ( q , r )}^{d} )$$

Q_{c} [symbol] {(q, r)∈Q_{G}×Q_{H}: (q, r) is a G-joint pair}

In some examples, an algorithm for PFSA may be as shown below:

Algorithm 1: GenESeSS

```
  Data: A sequence x over alphabet Σ, 0 < ε < 1
  Result: Stats set Q, transition map [symbol]  , and transition probability \tilde{π}
  /* Step One: Approximate [symbol]  sequence   */
  1 Let L = [log [symbol]   ];
  2 Calculate the derivative heap [symbol] [symbol]     equaling { [symbol]   : y is a sub-sequence of x with |y| [symbol]   L};
  3 Let [symbol]  be the convex hull of D [symbol]
  4 Select x [symbol]   with [symbol]   being a vertex of [symbol]  and has the highest frequency in x:
  /* Step Two: Identify transition structure   */
  5 Initialize Q = {q₀};
  6 Associate to q₀ the sequence identifier [symbol]  = x₀ and the probability vector [symbol]  ;
  7 Let \tilde{Q} be the set of stetes that are just added and initialize it to be Q;
  8 while \tilde{Q} ≠ [symbol]  do
  9  |  Let Q_{new} = [symbol]  be the set of new states;
10  |  for (q, σ) ∈ \tilde{Q} × Σ do
11  | | Let x = [symbol]   and d = [symbol]  ;
12  | | if ||d − d [symbol]   < [symbol]  for some [symbol]  ∈ Q then
13  | | | Let [symbol]  (q, σ) = q [symbol]
14  | | else
15  | | | Let Q_{new} = Q_{new} ∪ (Q_{new}) and Q = Q ∪ (q_{new});
16  | | | Associate to q_{new} the sequence identifier [symbol]  = xσ and the probability vector d [symbol]   = d;
17  | | | Let [symbol]  (q, σ) = q_{new};
18  |  Let \tilde{Q} = Q_{new};
19 Take a strongly connected subgraph of the labeled directed graph defined by Q and [symbol]  and denote the
    vertex set of the subgraph again by Q;
  /* Step Three: Identify transition probability   */
20 Initialize counter N [q, σ] for each pair (q, σ) ∈ Q × Σ;
21 Choose a random starting state q ∈ Q;
22 for σ ∈ x do
23  |  Let N [q, σ] = N [q, σ] + 1;
24  |  Let q = [symbol]  (q, σ);
25 Let \tilde{π}(q) = [[(N [q, σ]) [symbol]   ]];
26 return Q, [symbol]  , \tilde{π}; [symbol]
indicates data missing or illegible when filed
```

The algorithm shown above may be referenced as GenESess for Generator Extraction Using Self-Similar Semantics. The GenESess algorithm may take a sequence x as an input and a hyperparameter c and output a PFSA in following three steps. In a first step, the GenESess algorithm may approximate an almost synchronizing sequence, as described herein. In the next second step, the GenESess algorithm may identify transition structure of the PFSA and calculate the transition probabilities of the PFSA.

It should be understood that the algorithm for inferring PFSA shown above is for example only, other algorithms may also be used. From the generated PFSA algorithm, a sequence likelihood defect corresponding to occurring of a particular event based on a patient's health histories or EHRs in accordance with example theorems described below in view of the entropy rate and Kullback-Leibler (KL) divergence. The KL divergence is a non-symmetric metric measuring the relative entropy, or difference in information represented by two distributions. In other words, the KL divergence corresponds with a distance between two data distributions showing how different the two distributions are from each other. Entropy rate and KL divergence are described below using their respective equations.

An entropy rate of PFSA as described in the present disclosure may be an entropy rate of a stochastic process generated by the PFSA, and the KL divergence may be KL divergence between two stochastic processes generated by the PFSA. In some example, an entropy rate may be presented as:

$$𝒦 ( G ) = - {lim}_{d \to ∞} \frac{1}{d} {\sum }_{x ∈ {Σ}^{n}} p ( x ) log p ( x )$$

And the KL divergence may be presented as:

$${𝒟}_{KL} ( G \vert \vert H ) = {lim}_{d \to ∞} \frac{1}{d} {\sum }_{x ∈ {Σ}^{n}} {p}_{G} ( x ) log \frac{{p}_{G} ( x )}{{p}_{H} ( x )}$$

A following theorem may support the entropy rate and the KL divergence, as described herein, above.

For a PFSA G that is defined by the quadruple (Q, Σ, δ, \tilde{π}), an entropy rate [symbol] (G) may be:

$$ℋ ( G ) = {\sum }_{q ∈ Q} {p}_{G} ( q ) \cdot h \{ \tilde{π} ( q ) \}$$

In the above equation, h(v) is a based-2 entropy of the probability vector V.

KL divergence for two PFSA G and H, where:

G=(Q_{G}, Σ, δ_{G}, \tilde{π}_{G}), and

H=( [symbol] , Σ, [symbol] , [symbol] )

In addition, μ_{G} being absolutely continuous with respect to Σ_{H}, and Q_{c} being a set of G-joint pairs of states, the KL divergence may be:

$${𝒟}_{KL} ( G \vert \vert H ) = {\sum }_{( q , r ) ∈ {Q}_{c}} {p}_{G} ( q , r ) {D}_{KL} ( {\tilde{π}}_{G} ( q ) \vert \vert {\tilde{π}}_{H} ( τ ) )$$

### Log-Likelihood

In some examples, a log-likelihood of a PFSA G generating a sequence x may be presented as:

$$L ( x , G ) = - \frac{1}{d} log {p}_{G} ( x )$$

An algorithm for generating a log-likelihood of a PFSA G generating a sequence x in accordance with the above presentation or equation for the log-likelihood may be as shown below.

Algorithm 2: Log-likelihood

```
  Data: A PFSA G = (Σ, Q, δ, \tilde{π}) and a sequence x over alphabet Σ
  Result: Log-likelihood L(x, G) of G generating x
  1 Calculate the state transition matrix Π and observation \tilde{Π};
  2 Calculate the stationary distribution over states [symbol]  _{G} of G from Π;
  3 Calculate the stationary distribution of alphabet ϕ_{λ}^{T} = [symbol]   _{G}^{T}\tilde{Π};
  4 Initialize p by [symbol]  _{G} and q by ϕ_{λ};
  5 Let L = 0;
  6 for i from 1 to |x| do
  7  | Let σ be the i-th entry of x;
  8  | Let L = L − log q|_{σ};
  9  | Let p^{T} = [[p^{T}Γ_{σ}]] where Γ_{σ} is defined in 9;
10  | Let q^{T} = p^{T}\tilde{Π};
11 return L/|x|;
```

In some embodiments, convergence of log-likelihood for two PFSA G and H may be reduced PFSAs, and x∈Σ^{d} may be a sequence generated by PFSA G with d→∞, then:

L(x, H)→ [symbol] (G)+ [symbol] _{KL}(G∥H)

The above can be derived or proved based on the following theorem:

$${\sum }_{x ∈ {Σ}^{n}} {p}_{G} ( x ) log \frac{{p}_{G} ( x )}{{p}_{H} ( x )} = {\sum }_{x ∈ {Σ}^{d - 1}} {\sum }_{σ ∈ Σ} {p}_{G} ( x ) {p}_{G} ( x ) {\tilde{π}}_{G} {\vert}_{σ} log \frac{{p}_{G} ( x ) {p}_{G} ( x ) {\tilde{π}}_{G} {\vert}_{σ}}{{p}_{H} ( x ) {p}_{H} ( x ) {\tilde{π}}_{H} {\vert}_{σ}} = {\sum }_{x ∈ {Σ}^{d - 1}} {p}_{G} ( x ) log \frac{{p}_{G} ( x )}{{p}_{H} ( x )} + \underbrace{{\sum }_{x ∈ {Σ}^{d - 1}} {p}_{G} ( x ) {\sum }_{σ ∈ Σ} {p}_{G} ( x ) {\vert}_{σ} log \frac{{p}_{G} ( x ) {\tilde{π}}_{G} {\vert}_{σ}}{{p}_{H} ( x ) {\tilde{π}}_{H} {\vert}_{σ}}}_{{D}_{σ}}$$

And, therefore, by induction:

$${𝒟}_{KL} ( G \vert \vert H ) =. {lim}_{d \to ∞} \frac{1}{d} {\sum }_{i = 1}^{d} {D}_{i} ,$$

which based on Cesàro summation theorem may be represented as [symbol] _{KL}(G∥H)=lim_{d→∞}D_{d}.

For a sequence x=σ₁σ₂ . . . σₙ generated by a PFSA G, and an event x^{[i-1]} a truncation of an event x at the (i-1)-th symbol, then the following may be true.

$$- \frac{1}{n} {\sum }_{i = 1}^{n} log {p}_{H} ( {x}^{( i - 1 )} ) {\tilde{π}}_{H} {\vert}_{{σ}_{1}} = \underbrace{\frac{1}{n} {\sum }_{i = 1}^{n} log \frac{{p}_{G} ( {x}^{( i - 1 )} ) {\tilde{π}}_{G} {\vert}_{{σ}_{1}}}{{p}_{H} ( {x}^{( i - 1 )} ) {\tilde{π}}_{H} {\vert}_{{σ}_{1}}}}_{{A}_{n + 1}} - \underbrace{\frac{1}{n} {\sum }_{i = 1}^{n} log {p}_{G} ( {x}^{( i - 1 )} ) {\tilde{π}}_{G} {\vert}_{{σ}_{1}}}_{{B}_{n + 1}}$$

Because the stochastic process generated by the PFSA G is ergodic, then:

$${lim}_{n \to ∞} {A}_{x , n} = {lim}_{d \to ∞} {D}_{d} = {𝒟}_{KL} ( G \vert \vert H )$$

And

lim_{n→∞}B_{x, n}= [symbol] (G)

In the above, various important definitions and their corresponding equations, and algorithms to generate one or more PFSA models for each disease category may be generated using health histories or EHRs of a large number of patients. In the next steps after inferring various PFSA models, a pipeline including a network of various PFSA models may be trained. By way of a non-limiting example, the PFSA models may be gradient boosting classifiers, such as light gradient boosting machine (LGBM) classifiers. Each LGBM classifier of the LGBM classifiers may be individually trained and may operate on different categories of input features. Input features here may mean different diagnostic codes present in a patient's health history or EHRs. Exemplary input features may be as shown in “S-Table 6: Feature Definitions” of the Provisional Patent Application Ser. No. 63/353,236, the contents of which are incorporated herein in their entirety for all purposes.

In some examples, input features may be generated using raw data (e.g., health histories or EHRs) using one or more feature generators. The one or more feature generators may have parameters that need to be trained. Additionally, or alternatively, the one or more feature generators may include models that are required to be inferred. Inferencing of the one or more feature generators may be referenced herein as hyper-training. The hyper-training, as described and referenced in the present disclosure, may be different from hyper-parameters. Hyper-parameters include one or more variables having scalar values tuned by grid-search or via meta-heuristics to optimize classifiers, and hyper-training may produce one or more feature generators of different features in addition to a set of scalar values (or numbers).

In the following, hyper-training, training, and validation processes are described in detail.

In some examples, hyper-training and training may include trinary quantization of medical histories in which health histories or EHRs may be mapped into trinary disease-phenotype-specific data streams to enable generation of input features. Trinary quantization of medical histories may be performed, as described herein, by partitioning patient's health histories or EHRs into non-overlapping disease categories. Accordingly, those details are not repeated herein again for brevity.

Features used in the pipeline may be categorized as PFSA scores, prevalence scores, rare scores, sequence scores, and so on. In some examples, PFSA scores may be computed on the basis of inferred PFSA models. PFSA models may be inferred as described herein. The generation of the PFSA models from the trinary data streams may be a first hyper-training step, as described herein, that PFSA models are generated or inferred from patient data partitioned into non-overlapping disease categories. The PFSA scores may include positive log-likelihood and negative log-likelihood of a phenotype-specific quantized medical history being generated by the PFSA models for the positive cohort and the control cohort of sex-stratified patients, and corresponding sequence likelihood defects, as described in the present disclosure. The PFSA scores therefore encode the dynamics of the underlying processes, which may be sensitive to ordering and frequency of the codes at the resolution of the disease phenotypes. In some examples, disease phenotypes may include broad categories of diagnostics codes. One or more PFSA models may be generated for each disease category, each gender, and a positive cohort and a control cohort both.

In a second step of hyper-training, prevalence scores (or p-scores), which generally focus on individual diagnostic codes may be generated, and a dictionary of a ratio of relative prevalence of each diagnostic code in the positive cohort to the control cohort may be created. Relative prevalence of each diagnostic code may be relative to a set of all codes that are present in the particular cohort and for each gender.

Dictionaries of the ratio of relative prevalence of each diagnostic code may be used to map a diagnostic code to their p-scores, and also to aggregate other measures as mean, median, and variance for training an LGBM classified. In some examples, rare scores including a subset of p-scores that correspond with a particularly high p-score and/or a particularly low p-score may be identified. Sequence scores including mean, median, variance, and time since last occurrence, and so on, based on the trinary phenotype-specific sex-stratified health histories or EHRs may be identified or calculated. By way of an example, no hyper-training may be required for generation of the sequence features.

Accordingly, the training dataset may be further divided into three different subsets. The first subset may be used for hyper-training of the PFSA models and generating the p-score dictionary. The second subset may be used to train the LGBMs (in which an LGBM may correspond with each feature category). The third subset may be used to train the final LGBM that takes inputs from outputs of four LGBMs including an LGBM corresponding to each gender and a positive cohort and a control cohort.

In a validation, trinary mapping, features generated using the PFSA models, and the p-score dictionary may be used to calculate a raw risk score via the trained LGBM network of the pipeline. In some examples, the relative score may be determined in accordance with specificity and sensitivity trade-off as described herein.

The computer-implemented methods discussed herein may include additional, less, or alternate actions, including those discussed elsewhere herein. The methods may be implemented via one or more local or remote processors, transceivers, servers, and/or sensors, and/or via computer-executable instructions stored on non-transitory computer-readable media or medium.

Additionally, the computer systems discussed herein may include additional, less, or alternate functionality, including that discussed elsewhere herein. The computer systems discussed herein may include or be implemented via computer-executable instructions stored on non-transitory computer-readable media or medium.

In some embodiments, the system may be configured to implement machine learning, such that the neural network “learns” to analyze, organize, process, and/or validate data without being explicitly programmed. Machine learning may be implemented through machine learning (ML) methods and algorithms. In an exemplary embodiment, a machine learning (ML) module may be configured to implement ML methods and algorithms. In some embodiments, ML methods and algorithms may be applied to data inputs and generate machine learning (ML) outputs. Data inputs may include but are not limited to: analog and digital signals, sensor data, image data, video data, patient data, and the like. ML outputs may include but are not limited to: digital signals, medical diagnoses, segmented images, health care predictions and guidance, and the like. In some embodiments, data inputs may include certain ML outputs.

In some embodiments, at least one of a plurality of ML methods and algorithms may be applied, which may include but are not limited to: linear or logistic regression, instance-based algorithms, regularization algorithms, decision trees, Bayesian networks, cluster analysis, association rule learning, artificial neural networks, deep learning, recurrent neural networks, Monte Carlo search trees, generative adversarial networks, dimensionality reduction, and support vector machines. In various embodiments, the implemented ML methods and algorithms may be directed toward at least one of a plurality of categorizations of machine learning, such as supervised learning, unsupervised learning, and reinforcement learning.

In some embodiments, ML methods and algorithms may be directed toward supervised learning, which involves identifying patterns in existing data to make predictions about subsequently received data. Specifically, ML methods and algorithms directed toward supervised learning are “trained” through training data, which includes example inputs and associated example outputs. Based on the training data, the ML methods and algorithms may generate a predictive function which maps outputs to inputs and utilize the predictive function to generate ML outputs based on data inputs. The example inputs and example outputs of the training data may include any of the data inputs or ML outputs described above. For example, a ML module may receive training data comprising data associated with different patients and their corresponding outcomes, generate a model which maps the patient data to the outcome data, and recognize potential future outcomes for patients.

In some embodiments, ML methods and algorithms may be directed toward unsupervised learning, which involves finding meaningful relationships in unorganized data. Unlike supervised learning, unsupervised learning does not involve user-initiated training based on example inputs with associated outputs. Rather, in unsupervised learning, unlabeled data, which may be any combination of data inputs and/or ML outputs as described above, is organized according to an algorithm-determined relationship. In an exemplary embodiment, a ML module coupled to or in communication with the design system or integrated as a component of the design system receives unlabeled data, and the ML module employs an unsupervised learning method such as “clustering” to identify patterns and organize the unlabeled data into meaningful groups. The newly organized data may be used, for example, to extract further information about the potential classifications.

In some embodiments, ML methods and algorithms may be directed toward reinforcement learning, which involves optimizing outputs based on feedback from a reward signal. Specifically, ML methods and algorithms directed toward reinforcement learning may receive a user-defined reward signal definition, receive a data input, utilize a decision-making model to generate a ML output based on the data input, receive a reward signal based on the reward signal definition and the ML output, and alter the decision-making model so as to receive a stronger reward signal for subsequently generated ML outputs. The reward signal definition may be based on any of the data inputs or ML outputs described above. In an exemplary embodiment, a ML module implements reinforcement learning in a user recommendation application. The ML module may utilize a decision-making model to generate a ranked list of options based on user information received from the user and may further receive selection data based on a user selection of one of the ranked options. A reward signal may be generated based on comparing the selection data to the ranking of the selected option. The ML module may update the decision-making model such that subsequently generated rankings more accurately predict optimal constraints.

The computer-implemented methods discussed herein may include additional, less, or alternate actions, including those discussed elsewhere herein. The methods may be implemented via one or more local or remote processors, transceivers, servers, and/or sensors (such as processors, transceivers, and/or servers), and/or via computer-executable instructions stored on non-transitory computer-readable media or medium. Additionally, the computer systems discussed herein may include additional, less, or alternate functionality, including that discussed elsewhere herein. The computer systems discussed herein may include or be implemented via computer-executable instructions stored on non-transitory computer-readable media or medium.

As will be appreciated based on the foregoing specification, the above-described embodiments of the disclosure may be implemented using computer programming or engineering techniques including computer software, firmware, hardware or any combination or subset thereof, wherein the technical effect is to compile and optimize a variational quantum program for execution on a quantum processor. Any such resulting program, having computer-readable code means, may be embodied, or provided within one or more computer-readable media, thereby making a computer program product, (i.e., an article of manufacture), according to the discussed embodiments of the disclosure. The computer-readable media may be, for example, but is not limited to, a fixed (hard) drive, diskette, optical disk, magnetic tape, semiconductor memory such as read-only memory (ROM), and/or any transmitting/receiving medium such as the Internet or other communication network or link. The article of manufacture containing the computer code may be made and/or used by executing the code directly from one medium, by copying the code from one medium to another medium, or by transmitting the code over a network.

These conventional computer programs (also known as programs, software, software applications, “apps,” or code) include machine instructions for a conventional programmable processor and can be implemented in a high-level procedural and/or object-oriented programming language, and/or in assembly/machine language. As used herein, the terms “machine-readable medium” “computer-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The “machine-readable medium” and “computer-readable medium,” however, do not include transitory signals. The term “machine-readable signal” refers to any signal used to provide machine instructions and/or data to a programmable processor.

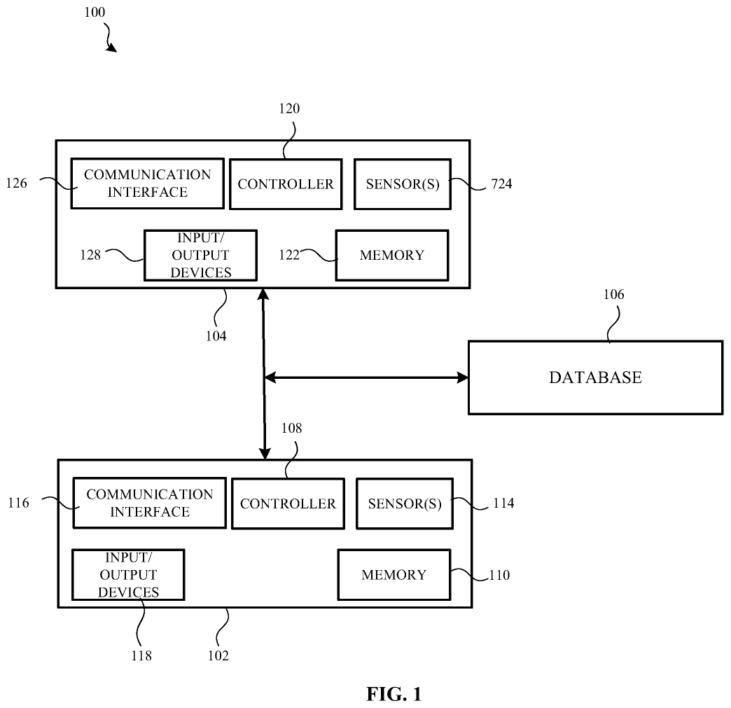

FIG. 1 is an example block diagram of a distributed computing system 100 including a client device 102 and an application server 104. The client device 102 may be communicatively coupled with the application server 104. By way of an example, the client device 102 may be a desktop, a laptop, a tablet, a smart phone, or any other user equipment. The client device 102 may be at a doctor's office, at a hospital, or in a primary care office, and so on. The client device 102 may include a controller 108, a memory 110, one or more sensors 114, a communication interface 116, and/or an input/output devices 118, and so on.

In some examples, the controller 108 may execute instructions stored in the memory 110. The controller 108 may include one or more processing units such as a central processing unit, a microprocessor, a microcontroller, a field programmable gate array (FPGA), and/or an application-specific integrated circuit (ASIC), and so on. The memory 110 may be a random-access memory (RAM), a static RAM (SRAM), a dynamic RAM (DRAM), a hard disk, a solid-state drive, a flash drive, and so on. The one or more sensors 114 may include an ultrasonic sensor, a light amplification by stimulated emission of radiation (Laser) sensor, X-ray, a heartrate monitor sensor, and so on, to measure vitals of a patient. The input/output device 118 may include one or more displays for outputting information to a user and a keyboard, a mouse, a touchpad screen, and so on, to receive user input.

The application server 104 may be a real-time data analyzing and classifying computer system that is configured to receive EHRs of a plurality of patients. The EHRs of the plurality of patients may be stored in a database 106. By way of an example, the database 106 may be a Truven Health MarketScan database or any other database from which electronic health records may be obtained. The application server 104 may configure PFSA models, for example, PFSA models for some predetermined number of disease categories. Further, the PFSA models may be generated for each gender, and for a positive cohort class and a control cohort class, as described herein. The PFSA models may be used to predict a risk for a patient to have a certain health condition based on previous electronic health records of the patient, without the patient to undergo any new laboratory blood work or any other tests.

The application server 104 may include a controller 120, a memory 122, one or more sensors 124, a communication interface 126, and/or an input/output devices 128, and so on. The controller 120 may execute instructions stored in the memory 122. The controller 120 may include one or more processing units such as a central processing unit, a microprocessor, a microcontroller, a field programmable gate array (FPGA), and/or an application-specific integrated circuit (ASIC), and so on, configured to implement various embodiments, as described in the present disclosure.

The memory 122 may be a random-access memory (RAM), a static RAM (SRAM), a dynamic RAM (DRAM), a hard disk, a solid-state drive, a flash drive, and so on. The one or more sensors 124 may include a capacitance-based sensor, an ultrasonic sensor, a light amplification by stimulated emission of radiation (Laser) sensor, X-ray, and so on. The input/output device 128 may include one or more displays for outputting information to a user and a keyboard, a mouse, a touchpad screen, and so on, to receive user input.

The communication interface 116 and the communication interface 126 provide communication and exchange of data between the client device 102 and the application server 104. The communication interface 116 and the communication interface 126 may include, for example, a local area network (LAN) or a wide area network (WAN) interface, dial-in-connections, cable modems, Internet connection, wireless, and special high-speed Integrated Services Digital Network (ISDN) lines. The communication interface 116 and the communication interface 126 may also provide communication and exchange of data with the database 106 to received EHRs of one or more patients.

In some examples, the client device 102 may receive EHRs of a patient as received from the database 106 to the application server 104 to determine a risk for the patient to acquire a particular health condition. The application server may use the generated PFSA models, as described herein, and predict a raw score of a patient's EHR to match with a PFSA model corresponding to a positive cohort and a control cohort for a gender of the patient. Based on the predicted raw score of the patient's EHR corresponding to the positive cohort and the control cohort for the gender of the patient, how likely the patient may acquire the particular health condition may be determined, and communicated to the client device 102.

Additionally, or alternatively, the application server 104 may transmit the generated PFSA models to the client device 102. The PFSA models may be installed or deployed on the client device 102. The client device 102 may then use deployed PF SA models to predict a raw score of a patient's EHR to match with a PF SA model corresponding to a positive cohort and a control cohort for a gender of the patient. Based on the predicted raw score of the patient's EHR corresponding to the positive cohort and the control cohort for the gender of the patient, how likely the patient may acquire the particular health condition may be determined.

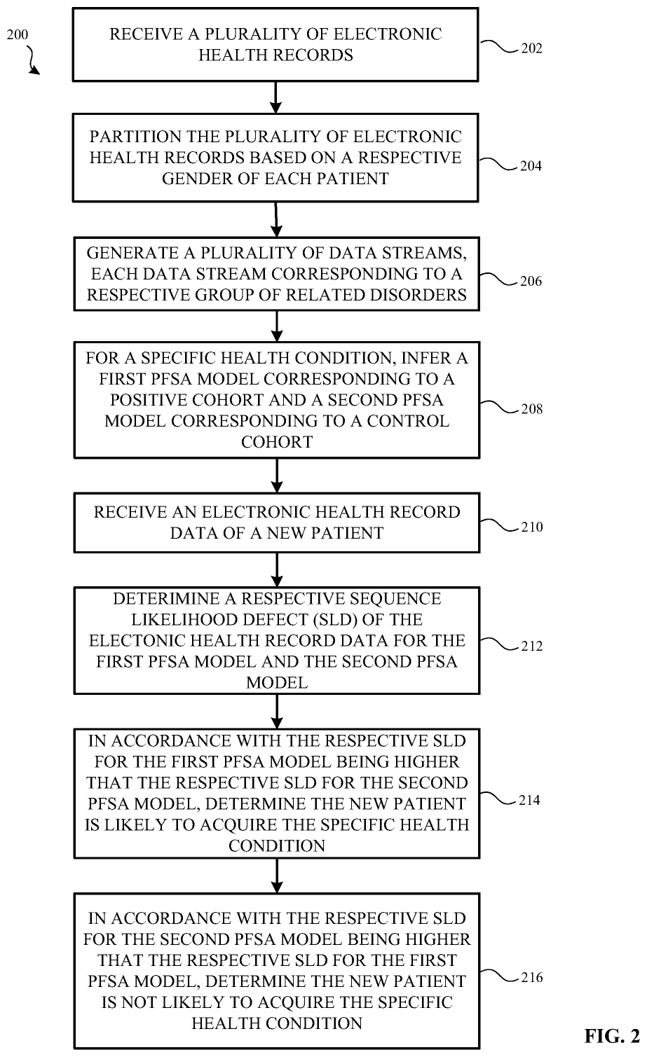

FIG. 2 is an example flow-chart 200 of method operations for predicting a specific health condition risk for a patient without requiring the patient undergoing any new laboratory work or new tests. At 202, a plurality of electronic health records stored in a database may be received by an application server. Each electronic health record of the plurality of electronic health records may correspond with a patient of a plurality of patients. In some examples, more than one electronic health record for a patient may also be received.

At 204, the plurality of electronic health records may be partitioned in a first set of plurality of electronic health records and a second set of plurality of health records. In other words, the plurality of electronic health records may be partitioned based on a gender of each patient of the plurality of patients. By way of an example, the first set of plurality of electronic health records may include electronic health records of males, and the second set of plurality of electronic health records may include electronic health records of females.

For each electronic health record of the first set of the plurality of health records and the second set of the plurality of health records, at 206, a plurality of data streams may be generated. Each data stream of the plurality of data streams may correspond with a respective group of related disorders. Each respective group of related disorders may be associated with a subset of diagnostic codes of a set of diagnostic codes. For example, the set of diagnostic codes may be ICD9 and/or ICD10 codes described herein.

At 208, in accordance with the generated data streams corresponding to the respective group of related disorders, a first PFSA model corresponding to a positive cohort for a specific health condition and a second PFSA model corresponding to a control cohort for the specific health condition may be inferred or generated. By way of a non-limiting example, the first PFSA model corresponding to the positive cohort may be generated from EHRs of patients having a diagnostic code from the set of diagnostic code that is associated with the specific health condition, and the second PFSA model corresponding to the control cohort may be generated from EHRs of patients who do not have the diagnostic code associated with the specific health condition. The generated PFSA models corresponding the respective group of related disorders may then be used or applied to predict a likelihood of another patient to acquire the specific health condition without any new laboratory work or new tests.

At 210, an electronic health record data of a new patient may be received, and based on the received electronic health record data of the new patient, at 212, a respective sequence likelihood defect of the electronic health record data to match the first PFSA model and the second PFSA model for the specific health condition may be determined. By way of a non-limiting example, the respective sequence likelihood defect of the electronic health record data to match the first PFSA model and the second PFSA model for the specific health condition may be determined using Kullbeck-Leibler (KL) divergence, as described herein.

At 214, based on the determined respective sequence likelihood defect of the electronic health record data to match the first PFSA model and the second PFSA model for the specific health condition, and the respective sequence likelihood defect of the electronic health record data to match the first PFSA model being higher than the respective sequence likelihood defect of the electronic health record data to match the second PFSA model, it may be determined the new patient has a higher likelihood to acquire the specific health condition.

At 216, based on the determined respective sequence likelihood defect of the electronic health record data to match the first PFSA model and the second PFSA model for the specific health condition, and the respective sequence likelihood defect of the electronic health record data to match the second PFSA model being higher than the respective sequence likelihood defect of the electronic health record data to match the first PFSA model, it may be determined the new patient has a lower likelihood to acquire the specific health condition.

In some embodiments, a plurality of features including PFSA score, p-scores, rare scores, and/or sequence scores corresponding to the first PFSA model and/or the second PFSA model may be generated. The generated plurality of features may be used for training a gradient boosting classifier. The trained gradient boosting classifier may be used to calculate a raw risk score corresponding to the electronic health record data of the new patient. The raw risk score may be calculated corresponding to the first PFSA model and the second PFSA model.

Accordingly, whether the new patient will acquire the specific health condition (and to have a diagnostic code corresponding to the specific health condition) being added to the new patient's EHR in future may be predicted without requiring the new patient to undergo any new laboratory work or new tests.

This written description uses examples to disclose the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A computer-implemented method, comprising:
receiving a plurality of electronic health records stored in a database, each electronic health record of the plurality of electronic health records corresponds with a patient of a plurality of patients;
based on a gender of each patient of the plurality of patients, partitioning the plurality of electronic health records in a first set of plurality of electronic health records and a second set of plurality of health records, the first set of plurality of electronic health records including electronic health records of males, and the second set of plurality of electronic health records including electronic health records of females;
for each electronic health record of the first and second sets of the plurality of electronic health records:
generating a plurality of data streams, each data stream of the plurality of data streams corresponds to a respective group of related disorders, the respective group of related disorders associated with a subset of diagnostic codes of a set of diagnostic codes;
in accordance with the generated data streams corresponding to the respective group of related disorders, inferring a first probabilistic finite state automaton (PFSA) model corresponding to a positive cohort for a specific health condition and a second PFSA model corresponding to a control cohort for the specific health condition;
receiving an electronic health record data of a new patient;
determining a respective sequence likelihood defect of the electronic health record data to match the first PFSA model and the second PFSA model for the specific health condition;
in accordance with the respective sequence likelihood defect of the electronic health record data to match the first PFSA model being higher than the respective sequence likelihood defect of the electronic health record data to match the second PFSA model, determining that the new patient has a higher likelihood to acquire the specific health condition; and
in accordance with the respective sequence likelihood defect of the electronic health record data to match the second PFSA model being higher than the respective sequence likelihood defect of the electronic health record data to match the first PFSA model, determining that the new patient has lower likelihood to acquire the specific health condition.

2. The computer-implemented method of claim 1, wherein generating each data stream of the plurality of data streams corresponding to the respective group of related disorders includes:
generating a trinary time-series data stream including a value per each respective unit of time period, the value per each respective unit of time period including at least one of: 0, 1, or 2, wherein:
the value 0 corresponds with an observation that any diagnostic code is absent during a respective unit of time period,
the value 1 corresponds with an observation that a diagnostic code associated with the respective group of related disorders is present during the respective unit of time period, and
the value 2 corresponds with an observation that a diagnostic code not associated with the respective group of related disorders is present during the respective unit of time period.

3. The computer-implemented method of claim 1, wherein each electronic health record of the first set of the plurality of electronic health records or the second set of the plurality of electronic health records including a same or a different number of diagnostic codes.

4. The computer-implemented method of claim 1, wherein the respective sequence likelihood defect of the electronic health record data to match the first PFS A model and the respective sequence likelihood defect of the electronic health record data to match the second PFSA model for the specific health condition is determined using Kullbeck Leibler (KL) divergence.

5. The computer-implemented method of claim 1, further comprising:
generating a plurality of features including PFSA scores, p-scores, rare scores, and sequence scores corresponding to the first PFSA model and the second PFSA model.

6. The computer-implemented method of claim 5, further comprising:
in accordance with the generated plurality of features, training a gradient boosting classifier; and
based on the trained gradient boosting classifier, calculating a raw risk score corresponding to the electronic health record data of the new patient for the first PFSA model and the second PFSA model.

7. The computer-implemented method of claim 6, wherein:
the calculated raw risk score for the new patient for first PFSA model corresponds with the respective sequence likelihood defect corresponding to the first PFSA model; and
the calculated raw risk score for the new patient for second PFSA model corresponds with the respective sequence likelihood defect corresponding to the first PFSA model.

8. The computer-implemented method of claim 1, wherein:
the first PFSA model corresponding to the positive cohort is inferred from the electronic health records of the first and the second sets of the plurality of electronic health records including a diagnostic code corresponding to the specific health condition; and
the second PFSA model corresponding to the control cohort is inferred from the electronic health records of the first and the second sets of the plurality of electronic health records in which the diagnostic code corresponding to the specific health condition is absent.

9. The computer-implemented method of claim 1, wherein the specific health condition includes at least one of major adverse cardiac event following a total hip or knee replacement, Idiopathic pulmonary fibrosis, autism spectrum disorder, or Alzheimer's diseases and related dementia.

10. The computer-implemented method of claim 1, wherein a diagnostic code associated with the specific health condition is absent in the electronic health record data of the new patient.

11. A system, comprising:
at least one memory configured to store instructions;
at least one processor communicatively coupled with the at least one memory, the at least one processor configured to execute the stored instruction, which cause the at least processor to:
receive a plurality of electronic health records stored in a database, each electronic health record of the plurality of electronic health records corresponds with a patient of a plurality of patients;
based on a gender of each patient of the plurality of patients, partition the plurality of health records in a first set of plurality of electronic health records and a second set of plurality of health records, the first set of plurality of electronic health records including electronic health records of males, and the second set of plurality of electronic health records including electronic health records of females;
for each electronic health record of the first and second sets of the plurality of electronic health records:
generate a plurality of data streams, each data stream of the plurality of data streams corresponds to a respective group of related disorders, the respective group of related disorders associated with a subset of diagnostic codes of a set of diagnostic codes;
in accordance with the generated data streams corresponding to the respective group of related disorders, infer a first probabilistic finite state automaton (PFSA) model corresponding to a positive cohort for a specific health condition and a second PFSA model corresponding to a control cohort for the specific health condition;
receive an electronic health record data of a new patient;
determine a respective sequence likelihood defect of the electronic health record data to match the first PFSA model and the second PFSA model for the specific health condition;
in accordance with the respective sequence likelihood defect of the electronic health record data to match the first PFSA model being higher than the respective sequence likelihood defect of the electronic health record data to match the second PFSA model, determine that the new patient has a higher likelihood to acquire the specific health condition; and
in accordance with the respective sequence likelihood defect of the electronic health record data to match the second PFSA model being higher than the respective sequence likelihood defect of the electronic health record data to match the first PFSA model, determine that the new patient has lower likelihood to acquire the specific health condition.

12. The system of claim 11, wherein to generate each data stream of the plurality of data streams corresponding to the respective group of related disorders, the stored instructions further cause the at least one processor to:
generate a trinary time-series data stream including a value per each respective unit of time period, the value per each respective unit of time period including at least one of: 0, 1, or 2, wherein:
the value 0 corresponds with an observation that any diagnostic code of the set of diagnostic codes is absent during a respective unit of time period,
the value 1 corresponds with an observation that a diagnostic code of the set of diagnostic codes associated with the respective group of related disorders is present during the respective unit of time period, and
the value 2 corresponds with an observation that a diagnostic code of the set of diagnostic codes not associated with the respective group of related disorders is present during the respective unit of time period.

13. The system of claim 11, wherein each electronic health record of the first set of the plurality of electronic health records or the second set of the plurality of electronic health records including a same or a different number of diagnostic codes.

14. The system of claim 11, wherein the respective sequence likelihood defect of the electronic health record data to match the first PFSA model and the respective sequence likelihood defect of the electronic health record data to match the second PFSA model for the specific health condition is determined using Kullbeck Leibler (KL) divergence.

15. The system of claim 11, wherein the stored instructions further cause the at least one processor to:
generate a plurality of features including PFSA scores, p-scores, rare scores, and sequence scores corresponding to the first PFSA model and the second PFSA model.

16. The system of claim 15, wherein the stored instructions further cause the at least one processor to:
in accordance with the generated plurality of features, train a gradient boosting classifier; and
based on the trained gradient boosting classifier, calculate a raw risk score corresponding to the electronic health record data of the new patient for the first PFSA model and the second PFSA model.

17. The system of claim 16, wherein:
the calculated raw risk score for the new patient for first PFSA model corresponds with the respective sequence likelihood defect corresponding to the first PFSA model; and
the calculated raw risk score for the new patient for second PFSA model corresponds with the respective sequence likelihood defect corresponding to the first PFSA model.

18. The system of claim 11, wherein:
the first PFSA model corresponding to the positive cohort is inferred from the electronic health records of the first and the second sets of the plurality of electronic health records including a diagnostic code of the set of diagnostic codes corresponding to the specific health condition; and
the second PFSA model corresponding to the control cohort is inferred from the electronic health records of the first and the second sets of the plurality of electronic health records in which the diagnostic code corresponding to the specific health condition is absent.

19. The system of claim 11, wherein the specific health condition includes at least one of major adverse cardiac event following a total hip or knee replacement, Idiopathic pulmonary fibrosis, autism spectrum disorder, or Alzheimer's diseases and related dementia.

20. The system of claim 11, wherein a diagnostic code of the set of diagnostic codes associated with the specific health condition is absent in the electronic health record data of the new patient.
